(19) United States
(12) Patent Application Publication
(10) Pub. No.: US 2026/0272884 A1
(43) Pub. Date: Sep. 17, 2026

(54) POVORCITINIB FOR TREATMENT OF HIDRADENITIS SUPPURATIVA

(71) Applicant: Incyte Corporation, Wilmington, DE (US)

(72) Inventors: Kurt Andrew Brown, Narberth, PA (US); Leandro Luiz Dos Santos, King of Prussia, PA (US); Joslyn S. Kirby, Hummelstown, PA (US)

(21) Appl. No.: 19/565,907

(22) Filed: Mar. 13, 2026

Related U.S. Application Data
(60) Provisional application No. 63/772,700, filed on Mar. 16, 2025.
(60) Provisional application No. 63/773,330, filed on Mar. 17, 2025.
(60) Provisional application No. 63/880,721, filed on Sep. 12, 2025.

(51) Int. Cl. A61K 31/4155; A61P 17/00

(52) U.S. Cl. CPC A61K 31/4155; A61P 17/00

(57) ABSTRACT

The present application provides methods of treating hidradenitis suppurativa in a patient in need thereof, comprising administering to the patient a therapeutically effective amount of povorcitinib, or a pharmaceutically acceptable salt thereof.

### CROSS-REFERENCE TO RELATED APPLICATIONS

This application claims the benefit of priority to U.S. Provisional Application No. 63/880,721 filed on Sep. 12, 2025, U.S. Provisional Application No. 63/773,330, filed on Mar. 17, 2025, and U.S. Provisional Application No. 63/772,700, filed on Mar. 16, 2025, the contents of each of which are hereby incorporated by reference.

### TECHNICAL FIELD

The present application provides methods for the treatment hidradenitis suppurativa (HS) using povorcitinib or a pharmaceutically acceptable salt thereof.

### BACKGROUND

Protein kinases (PKs) regulate diverse biological processes including cell growth, survival, differentiation, organ formation, morphogenesis, neovascularization, tissue repair, and regeneration, among others. Protein kinases also play specialized roles in a host of human diseases including cancer. Cytokines, low-molecular weight polypeptides or glycoproteins, regulate many pathways involved in the host inflammatory response to sepsis. Cytokines influence cell differentiation, proliferation and activation, and can modulate both pro-inflammatory and anti-inflammatory responses to allow the host to react appropriately to pathogens. Signaling of a wide range of cytokines involves the Janus kinase family (JAKs) of protein tyrosine kinases and Signal Transducers and Activators of Transcription (STATs). There are four known mammalian JAKs: JAK1 (Janus kinase-1), JAK2, JAK3 (also known as Janus kinase, leukocyte; JAKL; and L-JAK), and TYK2 (protein-tyrosine kinase 2).

Cytokine-stimulated immune and inflammatory responses contribute to pathogenesis of diseases: pathologies such as severe combined immunodeficiency (SCID) arise from suppression of the immune system, while a hyperactive or inappropriate immune/inflammatory response contributes to the pathology of autoimmune diseases (e.g., asthma, systemic lupus erythematosus, thyroiditis, myocarditis), and illnesses such as scleroderma and osteoarthritis (Ortmann, R. A., T. Cheng, et al. (2000) Arthritis Res 2(1): 16-32).

Deficiencies in expression of JAKs are associated with many disease states. For example, Jak1−/− mice are runted at birth, fail to nurse, and die perinatally (Rodig, S. J., M. A. Meraz, et al. (1998) Cell 93(3): 373-83). Jak2−/− mouse embryos are anemic and die around day 12.5 postcoitum due to the absence of definitive erythropoiesis.

The JAK/STAT pathway, and in particular all four JAKs, are believed to play a role in the pathogenesis of asthmatic response, chronic obstructive pulmonary disease, bronchitis, and other related inflammatory diseases of the lower respiratory tract. Multiple cytokines that signal through JAKs have been linked to inflammatory diseases/conditions of the upper respiratory tract, such as those affecting the nose and sinuses (e.g., rhinitis and sinusitis) whether classically allergic reactions or not. The JAK/STAT pathway has also been implicated in inflammatory diseases/conditions of the eye and chronic allergic responses.

Activation of JAK/STAT in cancers may occur by cytokine stimulation (e.g. IL-6 or GM-CSF) or by a reduction in the endogenous suppressors of JAK signaling such as SOCS (suppressor or cytokine signaling) or PIAS (protein inhibitor of activated STAT) (Boudny, V., and Kovarik, J., Neoplasm. 49:349-355, 2002). Activation of STAT signaling, as well as other pathways downstream of JAKs (e.g., Akt), has been correlated with poor prognosis in many cancer types (Bowman, T., et al. Oncogene 19:2474-2488, 2000). Elevated levels of circulating cytokines that signal through JAK/STAT play a causal role in cachexia and/or chronic fatigue. As such, JAK inhibition may be beneficial to cancer patients for reasons that extend beyond potential anti-tumor activity.

JAK2 tyrosine kinase can be beneficial for patients with myeloproliferative disorders, e.g., polycythemia vera (PV), essential thrombocythemia (ET), myeloid metaplasia with myelofibrosis (MMM) (Levin, et al., Cancer Cell, vol. 7, 2005: 387-397). Inhibition of the JAK2V617F kinase decreases proliferation of hematopoietic cells, suggesting JAK2 as a potential target for pharmacologic inhibition in patients with PV, ET, and MMM.

Inhibition of the JAKs may benefit patients suffering from skin immune disorders such as psoriasis, and skin sensitization. The maintenance of psoriasis is believed to depend on a number of inflammatory cytokines in addition to various chemokines and growth factors (JCI, 113:1664-1675), many of which signal through JAKs (Adv Pharmacol. 2000; 47:113-74).

Thus, new or improved agents which inhibit kinases such as JAKs are continually needed for developing new and more effective pharmaceuticals that are aimed at augmentation or suppression of the immune and inflammatory pathways, such as the treatment of hidradenitis suppurativa. This application is directed to that need and others.

### SUMMARY

Provided herein is a method of treating hidradenitis suppurativa comprising administering a therapy to a patient in need thereof, wherein the therapy comprises administering a therapeutically effective amount of a compound which is povorcitinib, or a pharmaceutically acceptable salt thereof, to the patient who has responded inadequately to an anti-TNF therapy.

Further provided herein is a method of treating hidradenitis suppurativa comprising administering a therapy to a patient in need thereof, wherein the therapy comprises administering a therapeutically effective amount of a compound which is povorcitinib, or a pharmaceutically acceptable salt thereof, to the patient who is intolerant to an anti-TNF therapy.

Further provided herein is a method of treating hidradenitis suppurativa comprising administering a therapy to a patient in need thereof, wherein the therapy comprises administering a therapeutically effective amount of a compound which is povorcitinib, or a pharmaceutically acceptable salt thereof, to the patient who is contraindicated to an anti-TNF therapy.

Further provided herein is a method of treating hidradenitis suppurativa comprising administering a therapy to a patient in need thereof, wherein the therapy comprises administering a therapeutically effective amount of a compound which is povorcitinib, or a pharmaceutically acceptable salt thereof, to the patient who has responded inadequately to conventional systemic hidradenitis suppurativa therapy.

Further provided herein is a method of treating hidradenitis suppurativa comprising administering a therapy to a patient in need thereof, wherein the therapy comprises administering a therapeutically effective amount of a compound which is povorcitinib, or a pharmaceutically acceptable salt thereof, to a patient who has responded inadequately to an anti-TNF therapy; and wherein the method results in a greater average improvement in HiSCR (%) relative to baseline in patients that were previously administered an anti-TNF therapy compared to patients that were not previously administered an anti-TNF therapy to treat hidradenitis suppurativa.

Further provided herein is a method of treating hidradenitis suppurativa comprising administering a therapy to a patient in need thereof, wherein the therapy comprises administering a therapeutically effective amount of a compound which is povorcitinib, or a pharmaceutically acceptable salt thereof, to a patient who is intolerant to an anti-TNF therapy; and wherein the method results in a greater average improvement in HiSCR (%) relative to baseline in patients that were previously administered an anti-TNF therapy compared to patients that were not previously administered an anti-TNF therapy to treat hidradenitis suppurativa.

Further provided herein is a method of treating hidradenitis suppurativa comprising administering a therapy to a patient in need thereof, wherein the therapy comprises administering a therapeutically effective amount of a compound which is povorcitinib, or a pharmaceutically acceptable salt thereof, to a patient who is contraindicated to an anti-TNF therapy; and wherein the method results in a greater average improvement in HiSCR (%) relative to baseline in patients that were previously administered an anti-TNF therapy compared to patients that were not previously administered an anti-TNF therapy to treat hidradenitis suppurativa.

Also provided herein is a compound which is povorcitinib, or a pharmaceutically acceptable salt thereof, for use in a method of treating provided herein. Also provided herein is use of a compound which is povorcitinib, or a pharmaceutically acceptable salt thereof, in the manufacture of a medicament for a method of treating provided herein.

### DETAILED DESCRIPTION

The present application provides, inter alia, methods of treating hidradenitis suppurativa comprising administering a therapy to a patient in need thereof, wherein the therapy comprises administering a therapeutically effective amount of a compound which is povorcitinib, which is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide, or a pharmaceutically acceptable salt thereof, having the structure shown below:

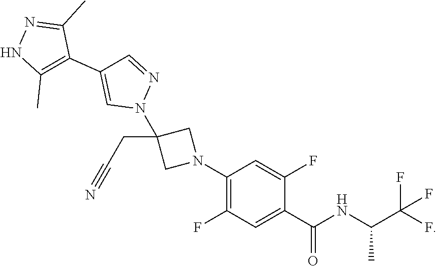 [SMILES: Cc1n[nH]c(C)c1-c1cnn(C2(CC#N)CN(c3cc(F)c(C(=O)N[C@@H](C)C(F)(F)F)cc3F)C2)c1]

Povorcitinib is described in U.S. Pat. No. 9,926,301, the entirety of which is incorporated herein by reference. Methods of treating hidradenitis suppurativa comprising the administration of JAK inhibitors such as povorcitinib are described in U.S. Pat. No. 11,304,949, the entirety of which is incorporated herein by reference.

The present application further provides a method of treating hidradenitis suppurativa comprising administering a therapy to a patient in need thereof, wherein the therapy comprises administering a therapeutically effective amount of a compound which is povorcitinib, or a pharmaceutically acceptable salt thereof, to the patient who has responded inadequately to an anti-TNF therapy.

The present application further provides a method of treating hidradenitis suppurativa comprising administering a therapy to a patient in need thereof, wherein the therapy comprises administering a therapeutically effective amount of a compound which is povorcitinib, or a pharmaceutically acceptable salt thereof, to the patient who is intolerant to an anti-TNF therapy.

The present application further provides a method of treating hidradenitis suppurativa comprising administering a therapy to a patient in need thereof, wherein the therapy comprises administering a therapeutically effective amount of a compound which is povorcitinib, or a pharmaceutically acceptable salt thereof, to the patient who is contraindicated to an anti-TNF therapy.

The present application further provides a method of treating hidradenitis suppurativa comprising administering a therapy to a patient in need thereof, wherein the therapy comprises administering a therapeutically effective amount of a compound which is povorcitinib, or a pharmaceutically acceptable salt thereof, to the patient who has responded inadequately to conventional systemic hidradenitis suppurativa therapy.

The present application further provides a method of treating hidradenitis suppurativa comprising administering a therapy to a patient in need thereof, wherein the therapy comprises administering a therapeutically effective amount of a compound which is povorcitinib, or a pharmaceutically acceptable salt thereof, to the patient who is intolerant to a conventional systemic hidradenitis suppurativa therapy.

The present application further provides a method of treating hidradenitis suppurativa comprising administering a therapy to a patient in need thereof, wherein the therapy comprises administering a therapeutically effective amount of a compound which is povorcitinib, or a pharmaceutically acceptable salt thereof, to the patient who is contraindicated to conventional systemic hidradenitis suppurativa therapy.

In some embodiments, the method is a method of reducing abscesses or inflammatory nodules resulting from hidradenitis suppurativa in the patient. In some embodiments, the method is a method of reducing the incidence of flares resulting from hidradenitis suppurativa in the patient. In some embodiments, the method is a method of reducing skin pain resulting from hidradenitis suppurativa in the patient. In some embodiments, the method is a method of reducing abscesses resulting from hidradenitis suppurativa in the patient. In some embodiments, the method is a method of reducing inflammatory nodules resulting from hidradenitis suppurativa in the patient.

The present application further provides a method of treating hidradenitis suppurativa in a patient that is intolerant to a conventional systemic hidradenitis suppurativa therapy, comprising (i) discontinuing administration of the conventional systemic hidradenitis suppurativa therapy to the patient, and (ii) administering a therapeutically effective amount of a compound which is povorcitinib, or a pharmaceutically acceptable salt thereof, to the patient. In some embodiments, the conventional systemic hidradenitis suppurativa therapy is an anti-TNF therapy. In some embodiments, the conventional systemic hidradenitis suppurativa therapy is adalimumab.

The present application further provides a method of treating hidradenitis suppurativa in a patient that is contraindicated to a conventional systemic hidradenitis suppurativa therapy, comprising (i) discontinuing administration of the conventional systemic hidradenitis suppurativa therapy to the patient, and (ii) administering a therapeutically effective amount of a compound which is povorcitinib, or a pharmaceutically acceptable salt thereof, to the patient. In some embodiments, the conventional systemic hidradenitis suppurativa therapy is an anti-TNF therapy. In some embodiments, the conventional systemic hidradenitis suppurativa therapy is adalimumab.

In some embodiments, the compound is povorcitinib free base.

In some embodiments, the pharmaceutically acceptable salt is povorcitinib phosphoric acid salt.

In some embodiments, the compound or the pharmaceutically acceptable salt is administered at a dosage from about 10 mg to about 100 mg on a free base basis. In some embodiments, the compound or pharmaceutically acceptable salt is administered at a dosage from about 15 mg to about 90 mg on a free base basis. In some embodiments, the compound or pharmaceutically acceptable salt is administered at a dosage from about 20 mg to about 90 mg on a free base basis. In some embodiments, the compound or pharmaceutically acceptable salt is administered at a dosage from about 30 mg to about 90 mg on a free base basis. In some embodiments, the compound or the pharmaceutically acceptable salt is administered at a dosage of about 45 mg or about 75 mg on a free base basis. In some embodiments, the compound or the pharmaceutically acceptable salt is administered at a dosage of about 45 mg on a free base basis. In some embodiments, the compound or the pharmaceutically acceptable salt is administered at a dosage of about 75 mg on a free base basis.

In some embodiments, the administering of the compound or the pharmaceutically acceptable salt is oral. In some embodiments, the compound or the pharmaceutically acceptable salt thereof is administered as a tablet. In some embodiments, the tablet comprises about 45 mg of the compound or the pharmaceutically acceptable salt thereof on a free base basis. In some embodiments, the tablet comprises about 75 mg of the compound or the pharmaceutically acceptable salt thereof on a free base basis. In some embodiments, the therapy comprises administering the compound or the pharmaceutically acceptable salt thereof once daily.

In some embodiments, the anti-TNF therapy is anti-TNF-alpha therapy. In some embodiments, the anti-TNF-alpha therapy is infliximab, etanercept, or adalimumab. In some embodiments, the anti-TNF-alpha therapy is adalimumab. In some embodiments, the patient received the anti-TNF therapy for at least one month. In some embodiments, the patient received the anti-TNF therapy for at least two months. In some embodiments, the patient received the anti-TNF therapy for at least three months. In some embodiments, the patient received the anti-TNF therapy for a period of one month to twelve months. In some embodiments, the patient received the anti-TNF therapy for a period of three months to twelve months. In some embodiments, the patient received the anti-TNF therapy for a period of three months to six months.

In some embodiments, the conventional systemic hidradenitis suppurativa therapy is an antibiotic. In some embodiments, the antibiotic is a topical antibiotic. In some embodiments, the topical antibiotic is clindamycin. In some embodiments, the antibiotic is a systemic antibiotic. In some embodiments, the systemic antibiotic is an oral antibiotic. In some embodiments, the systemic antibiotic is ciprofloxacin, clindamycin, dapsone, doxycycline, ertapenem, erythromycin, linezolid, metronidazole, minocycline, moxifloxacin, rifampin, or tetracycline.

In some embodiments, the conventional systemic hidradenitis suppurativa therapy is a topical treatment. In some embodiments, the topical treatment is azelaic acid, benzoyl peroxide, bleach baths, chlorhexidine, resorcinol, retinoids, steroids, or zinc pyrithione.

In some embodiments, the conventional systemic hidradenitis suppurativa therapy is a biologic drug. In some embodiments, the biologic drug is an anti-TNF-alpha biologic or an anti-IL 17A/anti-IL 17A/F biologic. In some embodiments, the biologic is selected from abatacept, adalimumab, anakinra, belimumab, bermekimab, bimekizumab, brodalumab, certolizumab pegol, dupilumab, efalizumab, etanercept, golimumab, guselkumab, imsidolimab, infliximab, iscalimab, ixekizumab, izokibep, lutikizumab, LY3041658, natalizumab, omalizumab, risankizumab, rituximab, secukinumab, sonelokimab, spesolimab, tocilizumab, ustekinumab, and vilobelimab.

In some embodiments, the conventional systemic hidradenitis suppurativa therapy is a systemic hormonal therapy. In some embodiments, the systemic hormonal therapy is ethinyl estradiol, cyproterone acetate, inasteride, metformin, or spironolactone.

In some embodiments, the conventional systemic hidradenitis suppurativa therapy is a JAK inhibitor. In some embodiments, the JAK inhibitor is selected from abrocitinib, baricitinib, brepocitinib, cerdulatinib, delgocitinib, deucravacitinib, filgotinib, ivarmacitinib, pacritinib, ritlecitinib, ropsacitinib, ruxolitinib, tofacitinib, and upadacitinib.

In some embodiments, the conventional systemic hidradenitis suppurativa therapy is a surgical procedure. In some embodiments, the surgical procedure is incision and drainage, deroofing, lesional ablation (electrosurgery), local excision, or laser therapy.

In some embodiments, the patient received the conventional systemic hidradenitis suppurativa therapy for at least one month. In some embodiments, the patient received the conventional systemic hidradenitis suppurativa therapy for at least two months. In some embodiments, the patient received the conventional systemic hidradenitis suppurativa therapy for at least three months. In some embodiments, the patient received the conventional systemic hidradenitis suppurativa therapy for a period of one month to twelve months. In some embodiments, the patient received the conventional systemic hidradenitis suppurativa therapy for a period of three months to twelve months. In some embodiments, the patient received the conventional systemic hidradenitis suppurativa therapy for a period of three months to six months.

In some embodiments, the method results in about 10% improvement in HiSCR (Hidradenitis Suppurativa Clinical Response). In some embodiments, the method results in about 20% improvement in HiSCR. In some embodiments, the method results in about 30% improvement in HiSCR. In some embodiments, the method results in about 40% improvement in HiSCR. In some embodiments, the method results in about 50% improvement in HiSCR.

In some embodiments, the method results in at least about 10% improvement in HiSCR (Hidradenitis Suppurativa Clinical Response). In some embodiments, the method results in at least about 20% improvement in HiSCR. In some embodiments, the method results in at least about 30% improvement in HiSCR. In some embodiments, the method results in at least about 40% improvement in HiSCR. In some embodiments, the method results in at least about 50% improvement in HiSCR.

In some embodiments, the method results in a decrease in the total number of abscesses resulting from hidradenitis suppurativa in the patient. In some embodiments, the method results in no increase in the total number of draining tunnels resulting from hidradenitis suppurativa in the patient. In some embodiments, the method results in a decrease in the incidence of flares resulting from hidradenitis suppurativa in the patient. In some embodiments, the method results in a decrease in skin pain resulting from hidradenitis suppurativa in the patient.

In some embodiments, the method results in a higher improvement in HiSCR compared with a patient who has previously been treated with an anti-TNF therapy. In some embodiments, the method results in a difference of greater than about 10 between HiSCR (%) of patients who are not treated with the compound or the pharmaceutically acceptable salt thereof (placebo) and HiSCR (%) patients who are treated with the compound or the pharmaceutically acceptable salt thereof. In some embodiments, the method results in a difference of greater than about 15 between HiSCR (%) of patients who are not treated with the compound or the pharmaceutically acceptable salt thereof (placebo) and HiSCR (%) patients who are treated with the compound or the pharmaceutically acceptable salt thereof. In some embodiments, the method results in a difference of greater than about 20 between HiSCR (%) of patients who are not treated with the compound or the pharmaceutically acceptable salt thereof (placebo) and HiSCR (%) patients who are treated with the compound or the pharmaceutically acceptable salt thereof. In some embodiments, the method results in a difference of greater than about 25 between HiSCR (%) of patients who are not treated with the compound or the pharmaceutically acceptable salt thereof (placebo) and HiSCR (%) patients who are treated with the compound or the pharmaceutically acceptable salt thereof. In some embodiments, the method results in a difference of greater than about 30 between HiSCR (%) of patients who are not treated with the compound or the pharmaceutically acceptable salt thereof (placebo) and HiSCR (%) patients who are treated with the compound or the pharmaceutically acceptable salt thereof.

In phase 3 clinical trial studies, patients who were previously treated with anti-TNF therapy (e.g., patients that demonstrated an inadequate response to the anti-TNF therapy, patients that were intolerant to the anti-TNF therapy, and/or patients who are contraindicated to the anti-TNF therapy) show about a 12%-25% greater HiSCR (%) response when treated with povorcitinib or a pharmaceutically acceptable salt thereof compared to placebo (in other words, a 12-25% improvement relative to baseline). This result is unexpected because patients who were not previously treated with anti-TNF therapy showed a 6%-10% greater HiSCR (%) response compared to placebo (in other words, a 6-10% improvement relative to baseline). Thus, the administration of povorcitinib, or a pharmaceutically acceptable salt thereof, provided a greater improvement in HiSCR (%) in patients who were previously administered an anti-TNF therapy than patients who were not previously administered an anti-TNF therapy to treat hidradenitis suppurativa.

In some embodiments, the claimed method results in a greater average improvement in HiSCR (%) relative to baseline in patients that were previously administered an anti-TNF therapy compared to patients that were not previously administered an anti-TNF therapy to treat hidradenitis suppurativa. In some embodiments, the claimed method results in a greater average improvement in HiSCR (%) relative to baseline in patients that are intolerant to anti-TNF therapy compared to patients that were not previously administered an anti-TNF therapy to treat hidradenitis suppurativa. In some embodiments, the method results in a greater average improvement in HiSCR (%) relative to baseline in patients that showed an inadequate response to anti-TNF therapy compared to patients that were not previously administered an anti-TNF therapy to treat hidradenitis suppurativa. In some embodiments, the method results in a greater average improvement in HiSCR (%) relative to baseline in patients that are contraindicated to anti-TNF therapy compared to patients that were not previously administered an anti-TNF therapy to treat hidradenitis suppurativa.

In some embodiments, the method provides an average improvement in HiSCR (%) relative to baseline that is at least about 10%. In some embodiments, the method provides an average improvement in HiSCR (%) relative to baseline that is at least about 11%. In some embodiments, the method provides an average improvement in HiSCR (%) relative to baseline that is at least about 12%. In some embodiments, the method provides an average improvement in HiSCR (%) relative to baseline that is at least about 11%, about 12%, about 13%, about 14%, about 15%, about 16%, about 17%, about 18%, about 19%, about 20%, about 21% about 22% about 23%, about 24%, or about 25%. As used herein, the term “baseline” refers to the HiSCR response (%) in a patient group that has been administered placebo rather than povorcitinib or a pharmaceutically acceptable salt thereof. Accordingly, the term “relative to baseline” refers to the difference between the HiSCR response (%) of patients that have been treated with povorcitinib or a pharmaceutically acceptable salt thereof and the HiSCR response (%) of patients that have been administered placebo.

In some embodiments, the patient is 18 years old or greater.

In some embodiments, the patient has moderate to severe hidradenitis suppurativa. In some embodiments, the patient has moderate hidradenitis suppurativa. In some embodiments, the patient has severe hidradenitis suppurativa.

In some embodiments, the compound can be an isotopically-labeled compound, or a pharmaceutically acceptable salt thereof. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms, such as —CD₃ being substituted for —CH₃).

One or more constituent atoms of the compounds described herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

Accordingly, in some embodiments, the compound is a compound wherein one or more hydrogen atoms in the compound are replaced by deuterium atoms, or a pharmaceutically acceptable salt thereof.

As used herein, the term “about” can mean±20% of the stated value, and includes more specifically values of ±10%, ±5%, ±2% and ±1% of the stated value.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “subject”, “individual” or “patient,” used interchangeably, refers to any animal, including mammals, preferably mice, rats, other rodents, rabbits, dogs, cats, swine, cattle, sheep, horses, or primates, and most preferably humans. In some embodiments, the “subject,” “individual,” or “patient” is in need of said treatment.

In some embodiments, the inhibitors are administered in a therapeutically effective amount. As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); or (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

As used herein, the term “preventing” or “treatment” refers to preventing the disease, condition or disorder in an individual who may be predisposed to the disease, condition or disorder but does not yet experience or display the pathology or symptomatology of the disease. In some embodiments, treating refers to inhibiting or ameliorating the disease. In some embodiments, treating is preventing the disease.

As used herein, “anti-TNF therapy” refers to a medication that blocks or inhibits the activity of tumor necrosis factor (TNF) or the physiologic response to TNF. Anti-TNF therapies can include biologics, such as monoclonal antibodies including but not limited to adalimumab, certolizumab pegol, golimumab, and infliximab. Anti-TNF therapies can further include small molecules, including but not limited to thalidomide, lenalidomide, and pomalidomide.

As used herein, the term “conventional systemic hidradenitis suppurativa therapy” refers to therapies that have the potential to inhibit or ameliorate hidradenitis suppurativa and/or symptoms thereof. A “conventional systemic hidradenitis suppurativa therapy” can refer to therapies that a physician or medical caregiver would deem suitable to treat hidradenitis suppurativa and/or symptoms thereof. In some embodiments, a “conventional systemic hidradenitis suppurativa therapy” refers to a therapy that is approved (e.g., by the United States Food & Drug Administration or the European Medicines Agency) for the treatment of hidradenitis suppurativa. In some embodiments, the conventional systemic hidradenitis suppurativa therapy is an oral antibiotic or a biologic therapy.

Examples of conventional systemic hidradenitis suppurativa therapies include, but are not limited to, the following: topical antibiotics (e.g., clindamycin); other topical treatments (e.g., azelaic acid, benzoyl peroxide, bleach baths/sodium hypochlorite, chlorhexidine, resorcinol, retinoids [isotretinoin, acitretin, alitretinoin], steroids, zinc pyrithione); systemic antibiotics (e.g., ciprofloxacin, clindamycin, dapsone, doxycycline, ertapenem, erythromycin, linezolid, metronidazole, minocycline, moxifloxacin, rifampin/rifampicin, tetracycline); systemic hormonal therapies (e.g., antiandrogen contraceptives [ethinyl estradiol, cyproterone acetate], finasteride, metformin, spironolactone); systemic or topical JAK inhibitors; other systemic therapies (e.g., apremilast, avacopan, azathioprine, colchicine, cyclosporine, methotrexate, retinoids [isotretinoin, acitretin, alitretinoin], steroids); biologics (e.g., adalimumab [Humira] or biosimilars) anti-TNF-α biologics (infliximab, etanercept); anti-IL 17A/anti-IL 17A/F biologics (secukinumab [Cosentyx], bimekizumab [Bimzelx], ixekizumab [Taltz]), and other biologics.

Examples of conventional systemic hidradenitis suppurativa therapies include surgical procedures such as, e.g., incision and drainage, deroofing, lesional ablation (electrosurgery), local excision, and laser therapy.

Examples of conventional systemic hidradenitis suppurativa therapies further include JAK inhibitory therapy, including but not limited to topical or systemic JAK inhibitors (e.g., inhibitors of JAK1, JAK2, JAK3, and TYK2). Examples of JAK inhibitors include, but are not limited to, the following:
abrocitinib (PF-4965842)
baricitinib
brepocitinib (PF-06700841)
cerdulatinib
delgocitinib
deucravacitinib (BMS-986165)
filgotinib
ivarmacitinib (ARQ-252; SHR-0302)
pacritinib
ritlecitinib (PF-06651600)
ropsacitinib (PF-06826647)
ruxolitinib
tofacitinib
upadacitinib

Examples of conventional systemic hidradenitis suppurativa therapy include prior biologic drug therapies such as, for example:
abatacept
adalimumab
anakinra
belimumab
bermekimab (JNJ-77474462)
bimekizumab
brodalumab
certolizumab pegol
dupilumab
efalizumab
etanercept
golimumab
guselkumab
imsidolimab (ANB019)
infliximab
iscalimab
ixekizumab
izokibep (ABY-035)
lutikizumab (ABT-981)
LY3041658
natalizumab
omalizumab
risankizumab
rituximab
secukinumab
sonelokimab (M1095)
spesolimab (BI-655130)
tocilizumab
ustekinumab
vilobelimab (IFX-1)

As used herein, “HiSCR” refers to the Hidradenitis Supparativa Clinical Response. HiSCR is a validated efficacy parameter that shows responsiveness to improvement in HS disease activity. HiSCR is described in detail in, for example, Kimball, A. B., J Eur Acad Dermatol Venereol. 2015 Jul. 22; 30(6):989-994.

As used herein, an “inadequate response” or “responded inadequately” can refer to a patient's response to a prior treatment for HS that did not result in sufficient inhibition or amelioration of the symptoms of HS. For example, an inadequate response can include instances such as the progression of a Hurley stage in the patient (e.g., the Hurley stage of at least one affected anatomical region has progressed from I to II, II to III, or I to III); the patient required at least one further intervention (e.g., incision and drainage or intralesional injection of a corticosteroid); the patient required an unplanned surgical procedure for HS; the patient was prescribed another therapy (including, but not limited to, a hormonal therapy, a biologic therapy, an oral steroid); the patient experienced pain interfering with activities of daily living (e.g., with unsatisfactory relief from over-the-counter analgesics); the patient experienced pain requiring opioids; the patient experienced drainage interfering with activities of daily living (e.g., requiring multiple dressing changes and/or changes of clothes daily); the patient experienced an increase in the number of anatomical regions affected by HS; or the patient experienced at least one new abscess or at least one new draining tunnel resulting from HS.

As used herein, “intolerance” or “intolerant,” when used with respect to a therapy for HS, refers to a therapy that has been discontinued in a patient because the therapy resulted in a significant adverse reaction in the patient. In some embodiments, a reaction is considered significant if the adverse reaction caused the patient discomfort and/or interrupted the patient's usual activities or function.

As used herein, “contraindicated,” when used with respect to a therapy for HS, refers to a therapy that has been discontinued in a patient because the therapy has been deemed not suitable for continued use in the patient, by, for example, a physician. In some embodiments, a therapy is contraindicated because the therapy resulted in a significant adverse reaction in the patient or the therapy provided an inadequate response in the patient. In some embodiments, a therapy is contraindicated because the therapy resulted in a significant adverse reaction in the patient. In some embodiments, a therapy is contraindicated because the therapy did not provide an adequate response in the patient.

### Combination Therapies

The methods described herein can further comprise administering one or more additional therapeutic agents. The one or more additional therapeutic agents can be administered to a patient simultaneously or sequentially.

In some embodiments, the additional therapeutic agent is an antibiotic. In some embodiments, the antibiotic is clindamycin, doxycycline, minocycline, trimethoprim-sulfamethoxazole, erythromycin, metronidazole, rifampin, moxifloxacin, dapsone, or a combination thereof. In some embodiments, the antibiotic is clindamycin, doxycycline, minocycline, trimethoprim-sulfamethoxazole, or erythromycin in combination with metronidazole. In some embodiments, the antibiotic is a combination of rifampin, moxifloxacin, and metronidazole. In some embodiments, the antibiotic is a combination of moxifloxacin and rifampin.

In some embodiments, the additional therapeutic agent is a retinoid. In some embodiments, the retinoid is etretinate, acitretin, tretinoin, adapalene, tazarotene, or isotretinoin.

In some embodiments, the additional therapeutic agent is a steroid. In some embodiments, the additional therapeutic agent is a corticosteroid. In some embodiments, the steroid is such as triamcinolone, dexamethasone, fluocinolone, cortisone, prednisone, prednisolone, or flumetholone.

In some embodiments, the additional therapeutic agent is an immunosuppressant. In some embodiments, the immunosuppressant is methotrexate or cyclosporin A. In some embodiments, the immunosuppressant is mycophenolate mofetil or mycophenolate sodium.

In some embodiments, the additional therapeutic agent is finasteride, metformin, or azelaic acid.

In some embodiments, the method further comprises administering an additional therapeutic agent selected from IMiDs, an anti-IL-6 agent, a hypomethylating agent, and a biologic response modifier (BRM).

Generally, a BRM is a substances made from living organisms to treat disease, which may occur naturally in the body or may be made in the laboratory. Examples of BRMs include IL-2, interferon, various types of colony-stimulating factors (CSF, GM-CSF, G-CSF), monoclonal antibodies such as abciximab, etanercept, infliximab, rituximab, trasturzumab, and high dose ascorbate.

In some embodiments, the hypomethylating agent is a DNA methyltransferase inhibitor. In some embodiments, the DNA methyltransferase inhibitor is selected from 5 azacytidine and decitabine.

Generally, IMiDs are as immunomodulatory agents. In some embodiments, the IMiD is selected from thalidomide, lenalidomide, pomalidomide, CC-11006, and CC-10015.

In some embodiments, the method further comprises administering an additional therapeutic agent selected from anti-thymocyte globulin, recombinant human granulocyte colony-stimulating factor (G CSF), granulocyte-monocyte CSF (GM-CSF), an erythropoiesis-stimulating agent (ESA), and cyclosporine.

In some embodiments, the method further comprises administering an additional JAK inhibitor to the patient. In some embodiments, the additional JAK inhibitor is ruxolitinib, barcitinib, tofacitinib, oclacitinib, filgotinib, gandotinib, lestaurtinib, momelotinib, bacritinib, PF-04965842, upadacitinib, peficitinib, fedratinib, cucurbitacin I, or CHZ868.

One or more additional pharmaceutical agents such as, for example, anti-inflammatory agents, immunosuppressants, as well as PI3Kδ, mTor, Bcr-Abl, Flt-3, RAF and FAK kinase inhibitors such as, for example, those described in WO 2006/056399, which is incorporated herein by reference in its entirety, or other agents can be used in combination with the compounds described herein for treatment of JAK-associated diseases, disorders or conditions. The one or more additional pharmaceutical agents can be administered to a patient simultaneously or sequentially.

Example Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491, all of which are incorporated herein by reference in their entirety.

Example suitable Flt-3 inhibitors include compounds, and their pharmaceutically acceptable salts, as disclosed in WO 03/037347, WO 03/099771, and WO 04/046120, all of which are incorporated herein by reference in their entirety.

Example suitable RAF inhibitors include compounds, and their pharmaceutically acceptable salts, as disclosed in WO 00/09495 and WO 05/028444, both of which are incorporated herein by reference in their entirety.

Example suitable FAK inhibitors include compounds, and their pharmaceutically acceptable salts, as disclosed in WO 04/080980, WO 04/056786, WO 03/024967, WO 01/064655, WO 00/053595, and WO 01/014402, all of which are incorporated herein by reference in their entirety.

In some embodiments, one or more of the compounds of the invention can be used in combination with one or more other kinase inhibitors including imatinib, particularly for treating patients resistant to imatinib or other kinase inhibitors.

In some embodiments, the additional therapeutic agent is fluocinolone acetonide (Retisert®), or rimexolone (AL-2178, Vexol, Alcon).

In some embodiments, the additional therapeutic agent is cyclosporine (Restasis®).

In some embodiments, the additional therapeutic agent is selected from Dehydrex™ (Holles Labs), Civamide (Opko), sodium hyaluronate (Vismed, Lantibio/TRB Chemedia), cyclosporine (ST-603, Sirion Therapeutics), ARG101(T) (testosterone, Argentis), AGR1012(P) (Argentis), ecabet sodium (Senju-Ista), gefarnate (Santen), 15-(s)-hydroxyeicosatetraenoic acid (15(S)-HETE), cevilemine, doxycycline (ALTY-0501, Alacrity), minocycline, iDestrin™ (NP50301, Nascent Pharmaceuticals), cyclosporine A (Nova22007, Novagali), oxytetracycline (Duramycin, MOLI1901, Lantibio), CF101 (2S,3S,4R,5R)-3,4-dihydroxy-5-[6-[(3-iodophenyl)methylamino]purin-9-yl]-N-methyl-oxolane-2-carbamyl, Can-Fite Biopharma), voclosporin (LX212 or LX214, Lux Biosciences), ARG103 (Agentis), RX-10045 (synthetic resolvin analog, Resolvyx), DYN15 (Dyanmis Therapeutics), rivoglitazone (DE011, Daiichi Sanko), TB4 (RegeneRx), OPH-01 (Ophtalmis Monaco), PCS101 (Pericor Science), REV1-31 (Evolutec), Lacritin (Senju), rebamipide (Otsuka-Novartis), OT-551 (Othera), PAI-2 (University of Pennsylvania and Temple University), pilocarpine, tacrolimus, pimecrolimus (AMS981, Novartis), loteprednol etabonate, rituximab, diquafosol tetrasodium (INS365, Inspire), KLS-0611 (Kissei Pharmaceuticals), dehydroepiandrosterone, anakinra, efalizumab, mycophenolate sodium, etanercept (Embrel®), hydroxychloroquine, NGX267 (TorreyPines Therapeutics), actemra, gemcitabine, oxaliplatin, L-asparaginase, or thalidomide.

In some embodiments, the additional therapeutic agent is an anti-angiogenic agent, cholinergic agonist, TRP-1 receptor modulator, a calcium channel blocker, a mucin secretagogue, MUC1 stimulant, a calcineurin inhibitor, a corticosteroid, a P2Y2 receptor agonist, a muscarinic receptor agonist, an mTOR inhibitor, another JAK inhibitor, Bcr-Abl kinase inhibitor, Flt-3 kinase inhibitor, RAF kinase inhibitor, and FAK kinase inhibitor such as, for example, those described in WO 2006/056399, which is incorporated herein by reference in its entirety. In some embodiments, the additional therapeutic agent is a tetracycline derivative (e.g., minocycline or doxycline). In some embodiments, the additional therapeutic agent binds to FKBP12.

In some embodiments, the additional therapeutic agent is an alkylating agent or DNA cross-linking agent; an anti-metabolite/demethylating agent (e.g., 5-flurouracil, capecitabine or azacitidine); an anti-hormone therapy (e.g., hormone receptor antagonists, SERMs, or aromotase inhibitor); a mitotic inhibitor (e.g. vincristine or paclitaxel); an topoisomerase (I or II) inhibitor (e.g. mitoxantrone and irinotecan); an apoptotic inducers (e.g. ABT-737); a nucleic acid therapy (e.g. antisense or RNAi); nuclear receptor ligands (e.g., agonists and/or antagonists: all-trans retinoic acid or bexarotene); epigenetic targeting agents such as histone deacetylase inhibitors (e.g. vorinostat), hypomethylating agents (e.g. decitabine); regulators of protein stability such as Hsp90 inhibitors, ubiquitin and/or ubiquitin like conjugating or deconjugating molecules; or an EGFR inhibitor (erlotinib).

In some embodiments, the additional therapeutic agent includes an antibiotic, antiviral, antifungal, anesthetic, anti-inflammatory agents including steroidal and non-steroidal anti-inflammatories, and anti-allergic agents. Examples of suitable medicaments include aminoglycosides such as amikacin, gentamycin, tobramycin, streptomycin, netilmycin, and kanamycin; fluoroquinolones such as ciprofloxacin, norfloxacin, ofloxacin, trovafloxacin, lomefloxacin, levofloxacin, and enoxacin; naphthyridine; sulfonamides; polymyxin; chloramphenicol; neomycin; paramomycin; colistimethate; bacitracin; vancomycin; tetracyclines; rifampin and its derivatives (“rifampins”); cycloserine; beta-lactams; cephalosporins; amphotericins; fluconazole; flucytosine; natamycin; miconazole; ketoconazole; corticosteroids; diclofenac; flurbiprofen; ketorolac; suprofen; cromolyn; lodoxamide; levocabastin; naphazoline; antazoline; pheniramine; or azalide antibiotic.

### Pharmaceutical Formulations and Dosage Forms

When employed as pharmaceuticals, the compounds of the invention can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

In some embodiments, the administration is topical. In some embodiments, the administration is topical administration to the skin.

In some embodiments, the administration is oral.

This invention also includes pharmaceutical compositions which contain, as the active ingredient, the compound of the invention or a pharmaceutically acceptable salt thereof, in combination with one or more pharmaceutically acceptable carriers (excipients). In some embodiments, the composition is suitable for topical administration. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier. In some embodiments, the composition comprises at least one compound described herein, or a pharmaceutically acceptable salt thereof, and at least one component selected from microcrystalline cellulose, lactose monohydrate, hydroxypropyl methylcellulose, and polyethylene oxide. In some embodiments, the composition comprises at least one compound described herein, or a pharmaceutically acceptable salt thereof, and microcrystalline cellulose, lactose monohydrate, and hydroxypropyl methylcellulose. In some embodiments, the composition comprises at least one compound described herein, or a pharmaceutically acceptable salt thereof, and microcrystalline cellulose, lactose monohydrate, and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel K00LV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from about 1 to about 1,000 mg, from about 1 mg to about 100 mg, from 1 mg to about 50 mg, and from about 1 mg to 10 mg of active ingredient, all on a free base basis. In some embodiments, the dosage is from about 1 mg to about 50 mg or about 1 mg to about 10 mg of active ingredient on a free base basis. In some embodiments, each dosage contains about 45 mg or 75 mg of the active ingredient on a free base basis. In some embodiments, each dosage contains about 45 mg of the active ingredient on a free base basis. In some embodiments, each dosage contains about 75 mg of the active ingredient on a free base basis. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions comprise from about 1 to about 1,000 mg, from about 1 mg to about 100 mg, from 10 mg to about 100 mg, and from about 20 mg to 80 mg of active ingredient, all on a free base basis. In some embodiments, the compositions comprise about 45 mg or about 75 mg of the active ingredient on a free base basis. In some embodiments, the compositions comprise about 45 mg of the active ingredient on a free base basis. In some embodiments, the compositions comprise about 75 mg of the active ingredient on a free base basis.

In some embodiments, the dosage of the compound, or a pharmaceutically acceptable salt thereof, is 45 mg or 75 mg on a free base basis. In some embodiments, the dosage of the compound, or a pharmaceutically acceptable salt thereof, is 45 mg on a free base basis. In some embodiments, the dosage of the compound, or a pharmaceutically acceptable salt thereof, is 75 mg on a free base basis.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present application. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present application.

The tablets or pills of the present application can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present application can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the invention. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present application can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the invention can further include one or more additional pharmaceutical agents, examples of which are listed hereinabove.

### Kits

The present application also includes pharmaceutical kits useful, for example, in the treatment and/or prevention of cytokine-related diseases or disorders, such as CRS, which include one or more containers containing a pharmaceutical composition comprising a therapeutically effective amount of a compound described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example A: Efficacy and Safety Study of Povorcitinib in Participants with Moderate to Severe Hidradenitis Suppurativa

### Study Overview

This example relates to a Phase 3, multicenter, randomized, parallel-group, placebo-controlled (PC), double-blind study in adult (18 years and older) participants with (i) moderate to severe HS (Hurley Stage II or III), (ii) HS lesions in at least two distinct anatomical areas, and (iii) a total abscess and inflammatory nodule count of at least 5 at screening and baseline. In addition, participants must have a history of failure of, intolerance to, or contraindication to an adequate course of conventional systemic therapy for HS. In particular, participants must have a documented history of inadequate response to a least a 3-month course of at least 1 conventional systemic therapy (e.g., oral antibiotic or biologic drug) for HS (or demonstrated intolerance to, or have a contraindication to, a conventional systemic therapy for their HS).

In the study, approximately 600 participants are randomized 1:1:1 to povorcitinib 45 mg once daily, povorcitinib 75 mg once daily, or placebo once daily for 12 weeks. Participants who complete the PC period may continue to a 42-week double-blind extension (EXT) period with povorcitinib 45 mg or 75 mg once daily.

Participants randomized to povorcitinib at baseline remain at their respective dose level (45 or 75 mg QD) throughout the PC and EXT periods, while participants randomized to placebo will receive povorcitinib 45 or 75 mg QD (1:1 ratio) at the Week 12 visit onward, after completion of the PC period. Treatment assignment during the PC and EXT periods are blinded to investigators and participants. Participants are screened for up to 28 days before the first dose of study drug; participants who are randomized in the study receive the first dose of study drug on Day 1, and the last dose within the PC period occurs at the Week 12 visit. The first dose of study drug within the 42-week EXT period occurs on the next day after the Week 12 visit; the last dose within the EXT period occurs on the day of the Week 54 visit (or last study drug administration known to the investigator or designee as reported in the EDC for patients who prematurely discontinue the study or are lost to follow-up). Participants who complete the 54-week treatment in this study may be eligible to participate in a separate extension study to further evaluate the safety and efficacy of the investigational product. For participants who do not participate in the separate extension study, a safety follow-up visit (EOS) is conducted approximately 30 (+7) days after EOT, or last administration of study drug (in case of premature discontinuation/early termination), for all participants.

The study drug is taken orally, preferably in the morning and at about the same time each day, with a full glass of water. The study drug can be taken with or without food. On the day of study drug visits at Week 3, Week 12, and Week 15, participants withhold study drug self-administration, as the dose is administered at the clinic after collection of PD and/or PK samples.

Povorcitinib is provided as 45 mg or 75 mg tablets; both strengths match each other in smell, taste, and appearance. Placebo tablets match the povorcitinib tablets in smell, taste, and appearance. No dose modifications are allowed during the study.

Lesion count assessments are performed to determine clinical efficacy endpoints; participants answer daily diary QoL questionnaires on skin pain, analgesic use, and itch and complete the study drug daily diary during the PC and EXT periods. Additional PRO questionnaires are answered by participants at the study visits. Participants are assessed for safety and tolerability throughout the study by monitoring the type, frequency, and severity of AEs and measuring vital signs and clinical laboratory parameters.

### Objectives and Endpoints

The primary objective is to evaluate the efficacy of povorcitinib. The endpoint is defined as the proportion of participants who achieve Hidradenitis Suppurativa Clinical Response (HiSCR) at Week 12. HiSCR is defined as at least a 50% reduction from baseline in the total abscess and inflammatory nodule count, with no increase from baseline in abscess or draining tunnel count.

A secondary objective is to evaluate the proportion of participants who achieve Hidradenitis Suppurativa Clinical Response 75 (HiSCR75) at Week 12. HiSCR75 is defined as at least a 75% reduction from baseline in the total abscess and inflammatory nodule count, with no increase from baseline in abscess or draining tunnel count.

An additional secondary objective is to evaluate the effect of povorcitinib in reducing the incidence of flares. The endpoint is defined by the proportion of participants with flare by Week 12. flare is defined as at least a 25% increase in the total abscess and inflammatory nodule count with a minimum increase of 2 relative to baseline.

An additional secondary objective is to evaluate the effect of povorcitinib in decreasing skin pain. The key secondary endpoint for regulatory purposes in the US/FDA/aligned countries if the proportion of participants with a ≥3-point decrease in Skin Pain Numeric Rating Scale (NRS) score at Week 12 among participants with baseline Skin Pain NRS score≥3. Participants with a Skin Pain score of at least 3 at baseline and who experience at least a 3-point decrease in Skin Pain score at Week 12, relative to baseline. Skin Pain is an 11-point NRS, ranging from 0 (no skin pain) to 10 (worst skin pain). A key secondary endpoint for regulatory purposes in EU/EMA/aligned countries is the proportion of participants who achieve Skin Pain NRS30 at Week 12 among participants with baseline Skin Pain NRS score≥3. Participants with a Skin Pain score of at least 3 at baseline and who achieve at Week 12 Skin Pain NRS30, defined as at least a 30% reduction and at least 1-unit reduction from baseline in the Skin Pain NRS.

### Prior Therapies

Participants must have a documented history of inadequate response to at least 3-month course of at least 1 conventional systemic therapy (oral antibiotic or biologic drug) for HS (or demonstrated intolerance to, or have a contraindication to, a conventional systemic therapy for treatment of their HS).

Examples of commonly used therapies for HS include, but are not limited to, the following: topical antibiotics (e.g., clindamycin); other topical treatments (e.g., azelaic acid, benzoyl peroxide, bleach baths/sodium hypochlorite, chlorhexidine, resorcinol, retinoids [isotretinoin, acitretin, alitretinoin], steroids, zinc pyrithione); systemic antibiotics (e.g., ciprofloxacin, clindamycin, dapsone, doxycycline, ertapenem, erythromycin, linezolid, metronidazole, minocycline, moxifloxacin, rifampin/rifampicin, tetracycline); systemic hormonal therapies (e.g., antiandrogen contraceptives [ethinyl estradiol, cyproterone acetate], finasteride, metformin, spironolactone); systemic or topical JAK inhibitors; other systemic therapies (e.g., apremilast, avacopan, azathioprine, colchicine, cyclosporine, methotrexate, retinoids [isotretinoin, acitretin, alitretinoin], steroids); biologics (e.g., adalimumab [Humira] or biosimilars) anti-TNF-α biologics (infliximab, etanercept); anti-IL 17A/anti-IL 17A/F biologics (secukinumab [Cosentyx], bimekizumab [Bimzelx], ixekizumab [Taltz]), and other biologics.

Examples of surgical procedures to treat HS include, but are not limited to incision and drainage, deroofing, lesional ablation (electrosurgery), local excision, and laser therapy.

Examples of prior JAK inhibitory therapy include any prior use, for any indication, of a topical or systemic JAK inhibitor (JAK1, JAK2, JAK3, TYK2). Examples of JAK inhibitors include, but are not limited to, the following:
abrocitinib (PF-4965842)
baricitinib
brepocitinib (PF-06700841)
cerdulatinib
delgocitinib
deucravacitinib (BMS-986165)
filgotinib
ivarmacitinib (ARQ-252; SHR-0302)
pacritinib
povorcitinib
ritlecitinib (PF-06651600)
ropsacitinib (PF-06826647)
ruxolitinib
tofacitinib
upadacitinib

Examples of prior biologic drug therapy include any prior use, for any indication, of an immunomodulatory biologic (or biosimilar) drug. Examples of immunomodulatory biologic drugs include, but are not limited to, the following:
abatacept
adalimumab
anakinra
belimumab
bermekimab (JNJ-77474462)
bimekizumab
brodalumab
certolizumab pegol
dupilumab
efalizumab
etanercept
golimumab
guselkumab
imsidolimab (ANB019)
infliximab
iscalimab
ixekizumab
izokibep (ABY-035)
lutikizumab (ABT-981)
LY3041658
natalizumab
omalizumab
risankizumab
rituximab
secukinumab
sonelokimab (M1095)
spesolimab (BI-655130)
tocilizumab
ustekinumab
vilobelimab (IFX-1)

If, after at least 3 months of conventional systemic therapy, any of the following have occurred, participants are considered to have had inadequate responses to the systemic therapy:
Progression of Hurley stage (ie, the Hurley stage of at least 1 affected anatomical region has progressed from I to II, II to III, or I to III)
Participant requires at least 1 intervention (eg, incision and drainage or intralesional injection of corticosteroid)
Participant required unplanned surgical procedure for HS
Participant was prescribed another therapy (hormonal, biologic, oral steroid)
Participant experiences pain interfering with activities of daily living, with unsatisfactory relief from over-the-counter analgesics (eg, ibuprofen or acetaminophen)
Participant experiences pain requiring opioids, including tramadol
Participant experiences drainage interfering with activities of daily living (eg, requires multiple dressing changes and/or changes of clothes daily)
Participant experiences an increase in the number of anatomical regions affected by HS
Participant experiences at least 1 new abscess or 1 new draining tunnel

The definition of intolerance to conventional systemic therapy (oral antibiotic or biologic drug) for HS is defined as when such therapy has been discontinued by a physician as a result of a significant adverse reaction to therapy administration. A reaction is considered significant if the adverse reaction was at least moderately severe (ie, the AE caused the participant discomfort and interrupts the participant's usual activities or function).

### Efficacy Assessments

Clinical evaluations of HS (lesion assessment/count and Hurley staging) are performed by an experienced evaluator.

HS lesion count is defined as a broad assessment of all the various skin “appearances” that a termed “lesions” in participants with HS. The lesion count addresses all relevant anatomical regions in each participant and is captured at all visits. The lesion count is used for calculation of various efficacy parameters, such as HiSCR, IHS4 (defined below), and flare incidence. The HS lesions counted in the study include abscess, inflammatory nodule, noninflammatory nodule, draining tunnel, and nondraining tunnel. The distinct anatomical areas that are considered for lesion assessment include face/neck, left axilla (armpit)/arm. Right axilla (armpit)/arm, left chest/breast and inframammary area, right chest/breast and inframammary area, abdomen, trunk/torso, left buttock (perianal), right buttock (perianal), left groin/thigh/leg, right groin/thigh/leg, genital area, and other areas.

The HiSCR is a validated endpoint that shows responsiveness to improvement in disease activity, simplifies the scoring process, and increases the sensitivity to detect certain HS-specific lesions. The HiSCR is defined by the status of 3 types of lesions: abscess, inflammatory nodules, and draining tunnels. The definition of response requires the 3 criteria below to be met, relative to baseline:
At least 50% decrease in abscess plus inflammatory nodule count; and
No increase in abscess count; and
No increase in draining tunnel count

HiSCR75 and HiSCR90 are also calculated by the sponsor as endpoints in the study. The only difference compared to HiSCR is the minimum percentage decrease in total abscess and inflammatory nodule count required to achieve response of 75% (HiSCR75) and 90% (HiSCR90).

Absolute and relative changes in the counts of the different types of HS lesions are also calculated.

The International HS Severity Score System (IHS4) is also used. The ISH4 is a composite, dynamic score and validated tool used to determine HS severity. It employs a weighted scale that sums the number of inflammatory nodules (multiplied by 1), the number of abscesses (multiplied by 2), and the number of draining tunnels (multiplied by 4). Although not used as an inclusion criteria for HS severity in this study, the total IHS4 score outlines the following disease categories: mild disease: ≤3 points; moderate disease: 4 to 10 points; severe disease: ≥11 points.

In this study, a flare is defined as at least 25% increase in the total abscess and inflammatory nodule count, relative to baseline; and an increase of at least 2 in the total abscess and inflammatory nodule count, relative to the baseline.

Hurley stage I is associated with abscess formation (single or multiple) without sinus tracts and cicatrization. Hurley stage II is associated with recurrent abscesses with tract formation and cicatrization; single or multiple, widely separated lesions. Hurley stage III is associated with diffuse or near-diffuse involvement or multiple interconnected tracts and abscesses across the entire area.

Participants answer various questionnaires to self-evaluate skin pain, analgesic use, itch, quality of life, dermatologic quality of life, fatigue, work productivity and activity impairment, and overall health.

### Statistics

The primary endpoint of this study is the proportion of participants who achieve HiSCR at Week 12. Participants will be randomized in a 1:1:1 ratio to 1 of 2 doses of povorcitinib (45 or 75 mg QD) or placebo and stratified by ANdT count (<11 and >11; approximately ≤50% of participants may have an ANdT count<11) and previous treatment of HS with a biologic drug. Based on the observed HiSCR rates at Weeks 8 and 12 reported in a Phase 2, randomized, dose-ranging study of povorcitinib, it is assumed that the HiSCR rate at Week 12 would be 48% for the povorcitinib 75 mg group, 45% for the povorcitinib 45 mg group, and 30% for the placebo group. Using the chi-square test, without continuity correction, at a 2-sided α=0.025, a sample size of 200 participants per group (with a total of 600 participants) will achieve approximately 93% power to detect a statistically significant difference between the povorcitinib 75 mg group and the placebo group and approximately 80.7% power to detect a statistically significant difference between the povorcitinib 45 mg and the placebo group.

### Preliminary Results

HiSCR response grouped by prior biologic use across two study groups is presented below. In all instances, the biologic was an anti-TNF biologic.

### Group 1:

| | | Povorcitinib, | Povorcitinib, |
|---|---|---|---|
| | Placebo | 45 mg QD | 75 mg QD |
| Biologic Experienced | (N = 73) | (N = 76) | (N = 74) |
| HiSCR response (%) | 21.9 | 34.2 | 37.8 |
| Rate difference | | 12.3 | 15.9 |
| Biologic Naïve | (N = 129) | (N = 128) | (N = 128) |
| HiSCR response (%) | 34.1 | 43.8 | 42.2 |
| Rate difference | | 9.6 | 8.1 |

### Group 2:

| | | Povorcitinib, | Povorcitinib, |
|---|---|---|---|
| | Placebo | 45 mg QD | 75 mg QD |
| Biologic Experienced | (N = 77) | (N = 80) | (N = 80) |
| HiSCR response (%) | 19.5 | 45.0 | 40.0 |
| Rate difference | | 25.2 | 20.5 |
| Biologic Naïve | (N = 126) | (N = 128) | (N = 128) |
| HiSCR response (%) | 34.1 | 40.6 | 43.8 |
| Rate difference | | 6.5 | 9.6 |

Thus, within a predefined subgroup of patients previously exposed to biologics, povorcitinib demonstrated greater differential efficacy (HiSCR50) when compared to placebo (nominal P-values):
Group 1: 45 mg: 34.2% vs. 21.9% [P=0.096]
   75 mg: 37.8% vs. 21.9% [P=0.037]
Group 2: 45 mg: 45.0% vs 19.5% [P=0.001]
   75 mg: 40.0% vs. 19.5% [P=0.005]

The above data demonstrates that povorcitinib administration provides a greater response (as determined by HiSCR) than placebo for the treatment of HS. The response is greater in those patients who were previously treated with a biologic. This greater response is unexpected, at least because patients who were previously treated with anti-TNF therapy (e.g., patients that demonstrated an inadequate response to the anti-TNF therapy, patients that were intolerant to the anti-TNF therapy, and/or patients who are contraindicated to the anti-TNF therapy) show about a 12%-25% greater HiSCR (%) response when treated with povorcitinib or a pharmaceutically acceptable salt thereof compared to placebo (in other words, a 12-25% improvement relative to baseline). This result is unexpected because patients who were not previously treated with anti-TNF therapy showed a 6%-10% greater HiSCR (%) response compared to placebo (in other words, a 6-10% improvement relative to baseline).

Additional preliminary results are presented below for the whole population groups (both the biologic experienced and biologic naïve populations combined).

HiSCR75 response across two study groups from the trial is presented below. HiSCR75 is defined as at least a 75% reduction from baseline in the total abscess and inflammatory nodule count, with no increase from baseline in abscess or draining tunnel count. In each instance, the data demonstrate a greater rate of HiSCR75 response in povorcitinib treatment groups compared to placebo.

### Group 1:

| | | |
|---|---|---|
| | Povorcitinib, | Povorcitinib, |
| Placebo | 45 mg QD | 75 mg QD |

Week 3

| | | | |
|---|---|---|---|
| HiSCR75 response (%) | 5.0 | 12.3 | 16.3 |
| Rate difference | | 7.3 | 11.3 |

Week 12

| | | | |
|---|---|---|---|
| HiSCR75 response (%) | 15.8 | 20.6 | 24.3 |
| Rate difference | | 4.8 | 8.5 |

### Group 2:

| | | |
|---|---|---|
| | Povorcitinib, | Povorcitinib, |
| Placebo | 45 mg QD | 70 mg QD |

Week 3

| | | | |
|---|---|---|---|
| HiSCR75 response (%) | 6.4 | 12.0 | 11.1 |
| Rate difference | | 5.6 | 4.7 |

Week 12

| | | | |
|---|---|---|---|
| HiSCR75 response (%) | 13.3 | 25.5 | 28.4 |
| Rate difference | | 12.2 | 15.1 |

Response as defined by a greater than or equal to 3-pt decrease in skin pain NRS is provided in the tables below for the two study groups.

### Study Group 1:

| | | |
|---|---|---|
| | Povorcitinib, | Povorcitinib, |
| Placebo | 45 mg QD | 75 mg QD |

Week 3

| | | | |
|---|---|---|---|
| Proportion of patients (%) | 5.4 | 12.1 | 10.8 |
| Rate difference | | 6.7 | 5.4 |

Week 12

| | | | |
|---|---|---|---|
| Proportion of patients (%) | 11.4 | 17.2 | 22.2 |
| Rate difference | | 5.8 | 10.8 |

### Stud Group 2:

| | | |
|---|---|---|
| | Povorcitinib, | Povorcitinib, |
| Placebo | 45 mg QD | 75 mg QD |

Week 3

| | | | |
|---|---|---|---|
| Proportion of patients (%) | 6.6 | 22.4 | 18.9 |
| Rate difference | | 15.8 | 12.3 |

Week 12

| | | | |
|---|---|---|---|
| Proportion of patients (%) | 9.3 | 29.1 | 22.0 |
| Rate difference | | 19.8 | 12.7 |

Response as defined by an at least 30% reduction and at least 1-unit reduction from baseline for participants with skin pain score of at least at baseline is presented for the two study groups below. The below results demonstrate a significant reduction of pain in each povorcitinib treatment group compared to placebo.

### Group 1:

| | | |
|---|---|---|
| | Povorcitinib, | Povorcitinib, |
| Placebo | 45 mg QD | 75 mg QD |

Week 3

| | | | |
|---|---|---|---|
| Proportion of patients (%) | 10.1 | 31.8 | 36.1 |
| Rate difference | | 21.7 | 26 |

Week 12

| | | | |
|---|---|---|---|
| Proportion of patients (%) | 22.1 | 31.8 | 34.8 |
| Rate difference | | 9.7 | 12.7 |

### Group 2:

| | | |
|---|---|---|
| | Povorcitinib, | Povorcitinib, |
| Placebo | 45 mg QD | 75 mg QD |

Week 3

| | | | |
|---|---|---|---|
| Proportion of patients (%) | 17.2 | 37.0 | 37.1 |
| Rate difference | | 19.8 | 19.9 |

Week 12

| | | | |
|---|---|---|---|
| Proportion of patients (%) | 17.9 | 40.6 | 37.7 |
| Rate difference | | 22.7 | 19.8 |

Response as defined by the percentage of participants with flare by week 12 is provided for the two study groups in the tables below. The data demonstrate a significant reduction in flares in each povorcitinib group compared to placebo.

### Group 1:

| | | Povorcitinib, | Povorcitinib, |
|---|---|---|---|
| | Placebo | 45 mg QD | 75 mg QD |
| | (N = 202) | (N = 204) | (N = 202) |
| Percentage of | 33.7 | 25.0 | 26.2 |
| participants with | | | |
| flare by Week 12 | | | |
| Difference in | | −8.7 (−17.5, 0.1) | −7.4 (−16.3, 1.5) |
| response rate | | | |

### Group 2:

| | | Povorcitinib, | Povorcitinib, |
|---|---|---|---|
| | Placebo | 45 mg QD | 75 mg QD |
| | (N = 203) | (N = 208) | (N = 208) |
| Percentage of | 33.5 | 21.6 | 20.7 |
| participants with | | | |
| flare by Week 12 | | | |
| Difference in | | −11.9 (−20.5, −3.4) | −12.9 (−21.3, −4.4) |
| response rate | | | |

In addition, preliminary results from the placebo-controlled period of the trial indicated that povorcitinib is well-tolerated at both dosages (45 mg and 75 mg), and no new safety signals were observed. A safety summary for the two study groups is provided below:

### Group 1:

| | Number (%) of participants with events | | | | | |
|---|---|---|---|---|---|---|
| | Placebo | | 45 mg | | 75 mg | |
| | (n = 202) | | (n = 204) | | (n = 202) | |
| Treatment-emergent | 106 | (52.5) | 121 | (59.3) | 134 | (66.3) |
| adverse event (TEAE) | | | | | | |
| Treatment-related | 32 | (15.8) | 43 | (21.1) | 60 | (29.7) |
| TEAE | | | | | | |
| Serious TEAE | 6 | (3.0) | 3 | (1.5) | 3 | (1.5) |
| Grade 3 or higher | 7 | (3.5) | 5 | (2.5) | 5 | (2.5) |
| TEAE | | | | | | |
| Fatal TEAE | 0 | (0.0) | 0 | (0.0) | 0 | (0.0) |
| TEAE leading to study | 3 | (1.5) | 4 | (2.0) | 7 | (3.5) |
| drug discontinuation | | | | | | |
| Most frequent TEAEs | | | | | | |
| Acne | 6 | (3.0) | 17 | (8.3) | 30 | (14.9) |
| Headache | 12 | (5.9) | 13 | (6.4) | 16 | (7.9) |
| Nasopharyngitis | 18 | (8.9) | 11 | (5.4) | 14 | (6.9) |
| Upper respiratory | 6 | (3.0) | 8 | (3.9) | 11 | (5.4) |

tract infection

### Group 2:

| | Number (%) of participants with events | | | | | |
|---|---|---|---|---|---|---|
| | Placebo | | 45 mg | | 75 mg | |
| | (n = 203) | | (n = 208) | | (n = 207) | |
| Treatment-emergent | 95 | (46.8) | 127 | (61.1) | 129 | (62.3) |
| adverse event (TEAE) | | | | | | |
| Treatment-related | 40 | (19.7) | 61 | (29.3) | 78 | (37.7) |
| TEAE | | | | | | |
| Serious TEAE | 4 | (2.0) | 4 | (1.9) | 4 | (1.9) |
| Grade 3 or higher | 5 | (2.5) | 8 | (3.8) | 6 | (2.9) |
| TEAE | | | | | | |
| Fatal TEAE | 0 | (0.0) | 0 | (0.0) | 0 | (0.0) |
| TEAE leading to study | 4 | (2.0) | 12 | (5.8) | 8 | (3.9) |
| drug discontinuation | | | | | | |
| Most frequent TEAEs | | | | | | |
| Acne | 11 | (5.4) | 18 | (8.7) | 23 | (11.1) |
| Headache | 7 | (3.4) | 14 | (6.7) | 16 | (7.7) |
| Nasopharyngitis | 9 | (4.4) | 10 | (4.8) | 15 | (7.2) |
| Upper respiratory | 6 | (3.0) | 12 | (5.8) | 12 | (5.8) |

tract infection

Provided below is a safety summary of particular adverse events in the two study groups. There was no evidence of major adverse cardiac events (MACE), malignancy, or thrombotic events at Week 12.

### Group 1

| | Number (%) of participants with events | | |
|---|---|---|---|
| Adverse events | Placebo | 45 mg | 75 mg |
| of special interest | (n = 202) | (n = 204) | (n = 202) |
| MACE | 0 (0.0) | 0 (0.0) | 0 (0.0) |
| Embolic/thromboembolic events | 0 (0.0) | 0 (0.0) | 0 (0.0) |
| Malignancies (any) | 0 (0.0) | 0 (0.0) | 0 (0.0) |
| Serious infections | 4 (2.0) | 0 (0.0) | 2 (1.0) |
| Opportunistic infections | 0 (0.0) | 0 (0.0) | 0 (0.0) |
| Herpes zoster | 0 (0.0) | 0 (0.0) | 1 (0.5) |
| HS exacerbations | 2 (1.0) | 1 (0.5) | 1 (0.5) |
| Hypersensitivity reactions | 3 (1.5) | 1 (0.5) | 0 (0.0) |

### Group 2

| | Number (%) of participants with events | | |
|---|---|---|---|
| Adverse events | Placebo | 45 mg | 75 mg |
| of special interest | (n = 203) | (n = 208) | (n = 207) |
| MACE | 0 (0.0) | 0 (0.0) | 0 (0.0) |
| Embolic/thromboembolic events | 0 (0.0) | 0 (0.0) | 0 (0.0) |
| Malignancies (any) | 2 (1.0) | 0 (0.0) | 0 (0.0) |
| Serious infections | 2 (1.0) | 0 (0.0) | 0 (0.0) |
| Opportunistic infections | 0 (0.0) | 1 (0.5) | 0 (0.0) |
| Herpes zoster | 0 (0.0) | 2 (1.0) | 2 (1.0) |
| HS exacerbations | 2 (1.0) | 2 (1.0) | 5 (2.4) |
| Hypersensitivity reactions | 2 (1.0) | 1 (0.5) | 1 (0.5) |

### Additional Interim Results

Additional interim results of the Phase 3 clinical trial are presented in this section. Among 329 patients with prior anti-TNF therapy (female, 63.5%; current smokers, 45.6%), Week 12 HiSCR50 was achieved by significantly more patients treated with povorcitinib vs placebo (45 mg, 39.0%; 75 mg, 36.3%; vs placebo, 21.4%; both P<0.025) using nonresponder imputation. Povorcitinib also demonstrated higher HiSCR75 (45 mg, 19.5%; 75 mg, 22.1%; vs placebo, 4.1%), HiSCR90 (45 mg, 10.2%, 75 mg, 13.3%; vs placebo, 3.1%), and ≥3-point decrease in Skin Pain NRS rates (45 mg, 19.6%; 75 mg, 18.2%; vs placebo, 7.5%).

Continued improvements were seen at Week 24 among povorcitinib-randomized and crossover patients (249 efficacy-evaluable patients): HiSCR50 (45 mg, 48.3%; 75 mg, 44.0%; placebo→45 mg, 48.6%; placebo→75 mg, 63.4%), HiSCR75 (45 mg, 33.7%; 75 mg, 27.4%; placebo→45 mg, 34.3%; placebo→75 mg, 34.1%), and HiSCR90 (45 mg, 20.2%; 75 mg, 14.3%; placebo→45 mg, 14.3%; placebo→75 mg, 24.4%). Improvements were also observed in ≥3-point decrease in Skin Pain NRS (45 mg, 33.3%; 75 mg, 34.4%; placebo→45 mg, 36.4%; placebo→75 mg, 39.4%). Both povorcitinib doses were generally well tolerated; serious adverse events and laboratory abnormalities were infrequent.

Thus, povorcitinib improved HS signs/symptoms through 24 weeks, including achievement of high-threshold HiSCR90 responses, in anti-TNF-experienced patients, which is a population that is likely to have greater disease burden and treatment challenges.

Various modifications of the invention, in addition to those described herein, will be apparent to those skilled in the art from the foregoing description. Such modifications are also intended to fall within the scope of the appended claims. Each reference cited in the present application, including all patent, patent applications, and publications, is incorporated herein by reference in its entirety.

1. A method of treating hidradenitis suppurativa comprising administering to a patient in need thereof a therapeutically effective amount of povorcitinib, or a pharmaceutically acceptable salt thereof, to the patient who has responded inadequately to an anti-TNF therapy.

2. A method of treating hidradenitis suppurativa comprising administering to a patient in need thereof a therapeutically effective amount of povorcitinib, or a pharmaceutically acceptable salt thereof, to the patient who is intolerant to an anti-TNF therapy.

3. A method of treating hidradenitis suppurativa comprising administering to a patient in need thereof, a therapeutically effective amount of povorcitinib, or a pharmaceutically acceptable salt thereof, to the patient who is contraindicated to an anti-TNF therapy.

4. A method of treating hidradenitis suppurativa comprising administering to a patient in need thereof a therapeutically effective amount of povorcitinib, or a pharmaceutically acceptable salt thereof, to the patient who has responded inadequately to conventional systemic hidradenitis suppurativa therapy.

5. The method of claim 1, wherein the method is a method of reducing abscesses or inflammatory nodules resulting from hidradenitis suppurativa in the patient.

6. The method of claim 1, wherein the method is a method of reducing the incidence of flares resulting from hidradenitis suppurativa in the patient.

7. The method of claim 1, wherein the method is a method of reducing skin pain resulting from hidradenitis suppurativa in the patient.

8-9. (canceled)

10. The method of claim 1, wherein the pharmaceutically acceptable salt is povorcitinib phosphoric acid salt.

11-12. (canceled)

13. The method of claim 1, wherein povorcitinib or the pharmaceutically acceptable salt is administered at a dosage of about 45 mg on a free base basis.

14. The method of claim 1, wherein povorcitinib or the pharmaceutically acceptable salt is administered at a dosage of about 75 mg on a free base basis.

15. The method of claim 1, wherein the administering of povorcitinib or the pharmaceutically acceptable salt is oral.

16. The method of claim 1, wherein povorcitinib or the pharmaceutically acceptable salt thereof is administered as a tablet.

17. The method of claim 16, wherein the tablet comprises about 45 mg of povorcitinib or the pharmaceutically acceptable salt thereof on a free base basis.

18. The method of claim 16, wherein the tablet comprises about 75 mg of povorcitinib or the pharmaceutically acceptable salt thereof on a free base basis.

19. The method of claim 1, wherein the method comprises administering povorcitinib or the pharmaceutically acceptable salt thereof once daily.

20. The method of claim 1, wherein the anti-TNF therapy is anti-TNF-alpha therapy.

21. The method of claim 20, wherein the anti-TNF-alpha therapy is infliximab, etanercept, or adalimumab.

22-28. (canceled)

29. The method of claim 4, wherein the conventional systemic hidradenitis suppurativa therapy is an antibiotic.

30-33. (canceled)

34. The method of claim 29, wherein the antibiotic is ciprofloxacin, clindamycin, dapsone, doxycycline, ertapenem, erythromycin, linezolid, metronidazole, minocycline, moxifloxacin, rifampin, or tetracycline.

35. The method of claim 4, wherein the conventional systemic hidradenitis suppurativa therapy is a topical treatment.

36. The method of claim 35, wherein the topical treatment is azelaic acid, benzoyl peroxide, bleach baths, chlorhexidine, resorcinol, retinoids, steroids, or zinc pyrithione.

37. The method of claim 4, wherein the conventional systemic hidradenitis suppurativa therapy is a biologic drug.

38. (canceled)

39. The method of claim 37, wherein the biologic drug is selected from abatacept, adalimumab, anakinra, belimumab, bermekimab, bimekizumab, brodalumab, certolizumab pegol, dupilumab, efalizumab, etanercept, golimumab, guselkumab, imsidolimab, infliximab, iscalimab, ixekizumab, izokibep, lutikizumab, LY3041658, natalizumab, omalizumab, risankizumab, rituximab, secukinumab, sonelokimab, spesolimab, tocilizumab, ustekinumab, and vilobelimab.

40-41. (canceled)

42. The method of claim 4, wherein the conventional systemic hidradenitis suppurativa therapy is a JAK inhibitor.

43. The method of claim 42, wherein the JAK inhibitor is selected from abrocitinib, baricitinib, brepocitinib, cerdulatinib, delgocitinib, deucravacitinib, filgotinib, ivarmacitinib, pacritinib, ritlecitinib, ropsacitinib, ruxolitinib, tofacitinib, and upadacitinib.

44-56. (canceled)

57. The method of claim 1, wherein the method results in a higher improvement in HiSCR compared with a patient who has previously been treated with an anti-TNF therapy.

58-62. (canceled)

63. The method of claim 1, wherein the patient is 18 years old or greater.

64. The method of claim 1, wherein the patient has moderate to severe hidradenitis suppurativa.

65. The method of claim 1, wherein the patient has moderate hidradenitis suppurativa.

66. The method of claim 1, wherein the patient has severe hidradenitis suppurativa.

67-79. (canceled)
